(19) United States
(12) Patent Application Publication
(10) Pub. No.: US 2010/0179513 A1
(43) Pub. Date: Jul. 15, 2010

(54) Apparatus, System, and Method for Providing a Catheter

(76) Inventor: Robert F. Hebeler, JR., Dallas, TX (US)

Correspondence Address: Patent Capital Group, 6119 McCommas Blvd, Dallas, TX, 75214, US

(21) Appl. No.: 12/353,033

(22) Filed: Jan. 13, 2009

(51) Int. Cl. A61M 25/18; A61M 25/098

(52) U.S. Cl. 604/529; 604/537

(57) ABSTRACT

An apparatus is provided and, in one example embodiment, includes a catheter coupled to a channel. The catheter includes a first set of holes in which materials may pass. The channel defines a pathway that extends along a portion of the catheter. The channel can interface with a second set of holes that are different from the first set of holes and access to the channel occurs via the second set of holes and not the first set of holes. The channel receives a substance that is delivered to an area close to or at the area in which the channel resides and the substance is delivered via the second set of holes. In more specific embodiments, the catheter includes a distal end that is rounded. In still other embodiments, the second set of holes are spaced equidistant from one another, and the channel interfaces with a syringe that delivers the substance.

### TECHNICAL FIELD OF THE INVENTION

This invention relates in general to medical procedures and, more particularly, to an apparatus, a system, and a method for providing a catheter.

### BACKGROUND OF THE INVENTION

While advances in medicine have offered creative and viable solutions for a number of illnesses, ailments, and diseases, some medical procedures have remained stagnant. These procedures almost appear crude, when contrasted with new technologies that have automated previous protocols and that have eliminated much of the discomfort experienced by patients.

For example, chest tube insertions can be problematic for a multitude of reasons. Typically, chest drains are placed in the pleural or mediastinal space to evacuate an abnormal collection of air or fluids that may have collected because of injury, disease, or surgical procedures. Any abnormal collection of fluid or air in the pleural space can compress the lung, causing it to collapse [either partially or fully], which can compromise ventilation and the mechanics of breathing. Insertion of a chest drain can allow drainage of the pleural space and help to restore and to maintain the negative pressure between the visceral and parietal pleural membranes: allowing a natural and a full expansion of the lung.

A chest drain is usually attached to an underwater seal drainage system. This acts as a one-way valve, allowing fluid and air to leave the pleural space during expiration and coughing, but which prevents it from being sucked back in during inspiration.

For general insertion procedures, patients are usually positioned on the bed and asked to move their arm [on the affected side] to be placed behind their head to expose the axillary area. Insertion of a chest drain is reported to be both painful and frightening: even under analgesia. In a given procedure, the skin [where the drain is going to be inserted] receives a local anesthetic. The most common position for chest tube insertion is the mid-axillary line. Blunt dissection of the subcutaneous tissue and muscle around the pleural cavity is achieved using forceps or the surgeon's finger.

Ideally, the position of the tube tip should be aimed towards the apex of the lung for a pneumothorax (air in the pleural space) or towards the base of the lung to remove fluid, for example, blood (haemothorax). A chest X-ray is performed after a chest drain has been inserted to verify its correct placement, the degree of re-expansion of the lung, and the residual pleural fluid and/or pneumothorax.

Missing from any such procedure are guidelines or protocols for the assessment and management of pain associated with chest drains. The medical community has acknowledged that chest drains can cause pain and that inadequate analgesia may inhibit both movement and lung expansion, but nothing has been done to address these issues. Thus, providing a safe, affordable, effective, and reliable mechanism for managing pain and discomfort in procedures like this (and others) presents a significant challenge for surgeons, device manufacturers, and hospitals, alike.

### BRIEF DESCRIPTION OF THE DRAWINGS

To provide a more complete understanding of the present invention and features and advantages thereof, reference is made to the following description, taken in conjunction with the accompanying figures, where like reference numerals represent like parts, in which:

FIG. 1 is a simplified schematic diagram that illustrates a catheter for addressing a multitude of pain management issues in accordance with an example embodiment of the present invention;

FIG. 2 is a simplified schematic view illustrating the supple nature of the example catheter of FIG. 1;

FIG. 3 is a simplified schematic diagram of a possible example tip for the catheter of FIG. 1; and

FIG. 4 is a simplified schematic diagram that illustrates one example procedure that may be performed with the assistance of the catheter of FIG. 1.

### DETAILED DESCRIPTION OF THE INVENTION

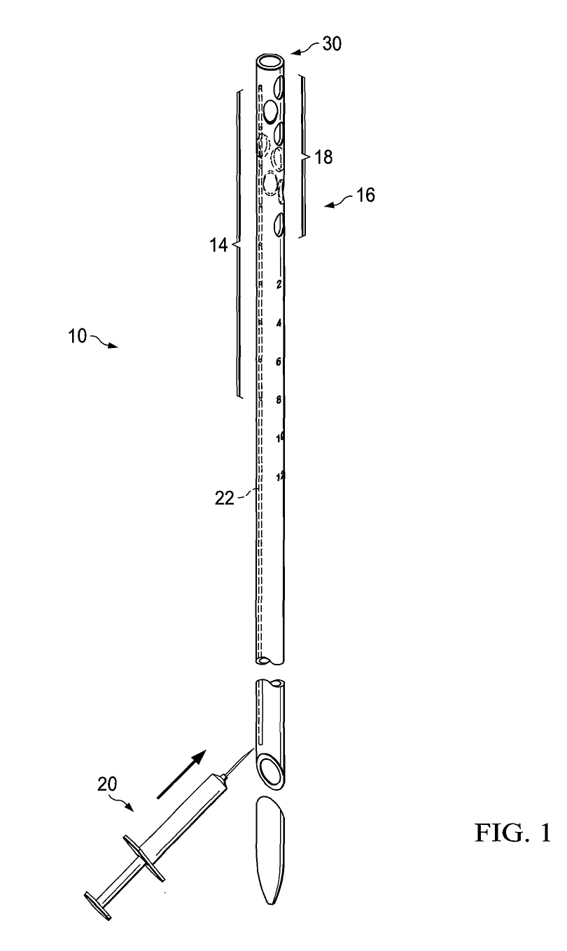

FIG. 1 illustrates a catheter 10 for delivering any type of substance (e.g., a fluid, solid, gas, or any combination thereof) to a targeted region. In one example, the substance is a fluid being delivered (e.g., a local anesthetic) to decrease the pain of indwelling tubes that are inserted into the body cavity. One example procedure that could use catheter 10 would involve a chest tube scenario.

Catheter 10 includes a number of delivery holes 14 that may be spaced apart at any suitable distance. In one example embodiment, delivery holes 14 are equidistant and spaced apart by 1 centimeter and stop at 8 centimeters on catheter 10, as is depicted. Catheter 10 also includes a radiopaque marker line 16. Catheter 10 includes a set of drain holes 18, which may be suitably positioned and configured (e.g., linear fashion, arbitrary, equidistant, alternating, etc.) to provide an outlet for materials to escape (e.g., fluid, air, and/or solid). In one example embodiment, drain holes 18 are configured as 270° outlets around catheter 10, where the holes are arbitrarily spaced around the circumference of catheter 10 to achieve a maximum flow through the holes when catheter 10 is positioned in a given surgical location. Both drain holes 18 and delivery holes 14 may be constructed as circles, hemispheres (i.e., ovals), half-circles, rectangles, triangles, squares, hexagons, or any other suitable shape, which may be based on particular patient or surgeon needs.

A channel 22 is provided within catheter 10, where channel 22 interfaces (or includes) delivery holes 14. Channel 22 is a small narrow pathway in which delivery holes 14 can be used for the delivery of any type of substance (e.g., a drug, pharmaceutical, gas, solid, or fluid). Channel 22 can be sealed such that access to channel 22 may occur only when its exterior is pierced (e.g., through a syringe, or a pump interface that is coupled to channel 22, etc.). A suitable interface may also be provided with channel 22 such that a quick connect (or an easy coupling) could occur between channel 22 and any other suitable element (e.g., for potentially delivering a substance to a targeted area).

In one example embodiment, there is an opposite marker line on catheter 10 for purposes of convenience and for orientation. In one example embodiment, catheter 10 also includes an optional syringe 20 that may be inserted into catheter 10 (e.g., at channel 22).

Syringe 20 can be optionally placed into channel 22, where delivery of a substance (e.g., a fluid, gas, solid, etc.) can be achieved. Catheter 10 may also be used for antibiotic irrigation of infected spaces in the body cavity. The medicine being delivered through channel 22 can exit catheter 10 at delivery holes 14, and not at drain holes 18. In this sense, the two sets of holes are isolated from each other. Syringe 20 could operate as a one-way valve, such as a Leur-Lok mechanism for the suitable delivery of any medicine or fluid. Instead of syringe 20, other delivery mechanisms may be used for the intermittent or continuous distribution of a substance to a targeted region of the body cavity. Syringe 20 represents simply one of a myriad of possible elements for the delivery of a substance along channel 22. Catheter 10 may also include a tip 30, which is depicted in detail in FIG. 3. Tip 30 is generally open at its end such that fluid from catheter 10 can exit or enter at this location.

In terms of the construction of catheter 10, any suitable material or composite may be used in constructing this element. In one example embodiment, a polyvinyl tube is used as part of catheter 10. In other embodiments, catheter 10 is constructed of kink-resistant polyvinyl chloride (PVC) tubing. In other embodiments, some suitable rubber, plastic, and/or metal alloy combination can be used in the construction of catheter 10. In certain cases, the metal alloy may provide better rigidity properties for catheter 10.

Turning to some of the specifications of catheter 10, this device could be constructed at any suitable length (e.g., 12, 14, 16, 18, 20, 22, 24, 26, 28, 32, 36, 40, 45 centimeters, etc.) or diameter. [Note that catheter 10 (in FIG. 1) has been truncated in terms of its proximal tip: simply for purposes of demonstrating that catheter 10 could extend farther down. This end could be simply cut or beveled based on particular surgical needs. Along similar lines, this proximal tip could be coupled to any number of mechanisms that assist in the aspiration, suction, and/or pressurized activities associated with catheter 10.] Turning to the opposite end, the distal tip of catheter 10, in one example, is rounded and curved such that it operates as a blunt, obturator instrument that minimizes poking and/or discomfort when inserted in the body cavity. This end can also help in guiding catheter 10 to its targeted region. This end is open in a preferred embodiment, but could be closed if the patient environment necessitates such a configuration.

The dimensions of catheter 10 could be varied considerably without departing from the scope of the present invention. Catheter 10 can be used in virtually any procedure where such a catheter could be beneficial. For example, catheter 10 could be used in post-operative drainage activities, after cardio-thoracic and thoracic surgery. Catheter 10 can be somewhat atraumatic due to its shape and drug delivery mechanism. In addition, catheter 10, with its rounded open distal end with smooth eyes, minimizes typical inflammation issues caused by other catheters.

In terms of the dimensions of the holes of catheter 10, any suitable diameter, spacing, length, width, and depth (or height) may be used and can be based on particular end user needs or specific elements to be addressed by catheter 10. Additionally, more holes may be provided, along with more channels provided to catheter 10. Although illustrated as linear, delivery holes 14 could be spaced differently and/or be configured to go around catheter 10 in a more circular or serpentine fashion. In some embodiments, delivery holes 14 could be spaced radially such that they are somewhat co-planar with one another. In such an example, channel 22 would extend up catheter 10 and then a second channel could be provided at that planar level, where the delivery would occur at that plane at which delivery holes 14 are located. In that example, the substance being sent along channel 22 would traverse the length of catheter 10 until reaching any location at which delivery holes 14 would reside.

It is imperative to note that all of the specifications and relationships outlined herein (e.g., spacing, height, width, length, hole diameter, number of holes, etc.) have only been offered for purposes of example and teaching only. Each of these data may be varied considerably without departing from the spirit of the present invention, or the scope of the appended claims. The specifications apply only to one non-limiting example and, accordingly, should be construed as such.

For purposes of teaching and discussion, it is useful to provide some overview as to the way in which thoracic catheters typically operate. The following foundational information may be viewed as a basis from which the present invention may be properly explained. Such information is offered earnestly for purposes of explanation only and, accordingly, should not be construed in any way to limit the broad scope of the present invention and its potential applications.

In many surgical procedures, an original incision is made and this can often occur proximate to, or at, the chest cavity. Subsequent to the initial procedure, a thoracic catheter is implemented to provide some type of drainage function. The thoracic catheter is typically inserted through an entry point of the body. The catheter is provided with both a distal end and a proximal end. In one general application, following intra-thoracic resections, drain tubes are typically placed to evacuate air and fluid from a targeted space. Drain tubes can also aid in the re-expansion of the lungs. The portion of the pleura external to the pulmonary pleura lines the inner surface of the chest wall, covers the diaphragm, and is reflected over the structures occupying the middle of the thorax. This portion is termed the parietal pleura and can be sensitive. The parietal pleura is attached to the wall of the thoracic cavity and innervated by the intercostal nerves. Thus, the parietal pleura is rich in pain receptors and the mechanical contact of the chest tube produces pleurisy or pain from inflammation of the pleura.

Procedures in this general area can be complicated and delicate, as the surgeon is often called on to position the catheter without a clear line of sight. Uniformly, the insertion of a catheter in the chest cavity causes significant discomfort. Catheter 10 can attenuate some of this pain in providing a viable alternative to crude procedures, which fail to account for this pain issue. In more specific embodiments, channel 22 (which may ride atop catheter 10 as a separate element, or be provided integral to catheter 10) can be used to deliver a substance (e.g., a local anesthesia) to a targeted region. Thus, channel 22 may be integrally coupled to catheter 22 or coupled to catheter 10 as a somewhat external (or independent) component that accompanies catheter 10. The substance can be delivered a single time, during movement or placement of catheter 10, intermittently, or in a constant infusion. Catheter 10 can be positioned with channel 22 adjacent to the chest wall, or positioned in other locations of the body cavity based on different procedures.

Shifting now to an example procedure that could be carried out by a surgeon, the following steps could be performed in conjunction with the use of catheter 10 for closed chest insertion. Initially, conscious sedation during the procedure is an option for those patients who are clinically stable. At a first step, a surgeon can examine the patient and assess the need for placement of a thoracostomy tube. At a second step, a surgeon can obtain a pre-procedure chest X-ray. At a third step, a surgeon can select a site for insertion (e.g., mid-axillary line, between 4th and 5th ribs, which is usually on a line lateral to the nipple). At a fourth step, the surgeon preps and drapes the area of insertion.

In addition, the surgeon can have the patient place the ipsilateral arm over the head to “open up” the ribs. At a fifth step, a surgeon can widely anesthetize area of insertion with a suitable medication (e.g., a 2% lidocaine dosage). The surgeon can infiltrate the skin, the muscle tissues, and down to the pleura. For catheter 10 insertion (at a sixth step), after infiltrating the insertion site with the local anesthetic, the surgeon can make a 1-2 centimeter incision through the skin and subcutaneous tissues between the 4th and 5th ribs. For incising in the chest wall, the surgeon can continue the incision through the intercostal muscles and down to the pleura.

At a seventh step, the surgeon can insert a clamp (e.g., a Kelly clamp) through the pleura and open the jaws widely, again parallel to the direction of the ribs to spread muscle fibers. At this point (and at an eighth step), a surgeon may or may not insert a finger through the incision and into the thoracic cavity: making sure he is feeling a lung (or empty space) and not a liver or a spleen. At a ninth step, the surgeon can grasp an end of catheter 10 with the forceps (convex angle towards ribs), and insert catheter 10 through the hole made in the pleura.

Use of catheter 10 empowers a surgeon to address a number of pain and discomfort issues. Any suitable substance (e.g., medications) can start being delivered through catheter 10 at the moment catheter 10 enters the body cavity. In other scenarios, channel 22 is preloaded such that it has medications already in it. These medications could be delivered through channel 22, where fluid would exit delivery holes 14. In other examples, the medication identified here is not delivered until catheter 10 is stabilized or secured in some fashion. In still other embodiments, medication is only delivered upon insertion and/or removal of catheter 10. In still other embodiments, time released medications are used (e.g., having certain half-lives, etc.) such that a surgeon is able to control the exact dosage of the medication being delivered. The important point is that the surgeon can, at any time period, begin delivery of a substance to a targeted site through delivery holes 14 via channel 22.

Returning to the next step in the procedure, after catheter 10 has entered the thoracic cavity, the surgeon can remove the Kelly clamp and manually advance catheter 10. The clamp can guide insertion of catheter 10. At a tenth step, catheter 10 can be sutured and/or taped in place. At this point, a medication could begin to be delivered (e.g., a constant dosage or perfusion) to a location via channels 20. This delivery could be achieved through a syringe or through a suitable pump. In addition, catheter 10 can be attached to a suction unit. As a final step, the surgeon can obtain a post procedure chest X-ray for placement confirmation. Catheter 10 may need to be advanced or withdrawn slightly.

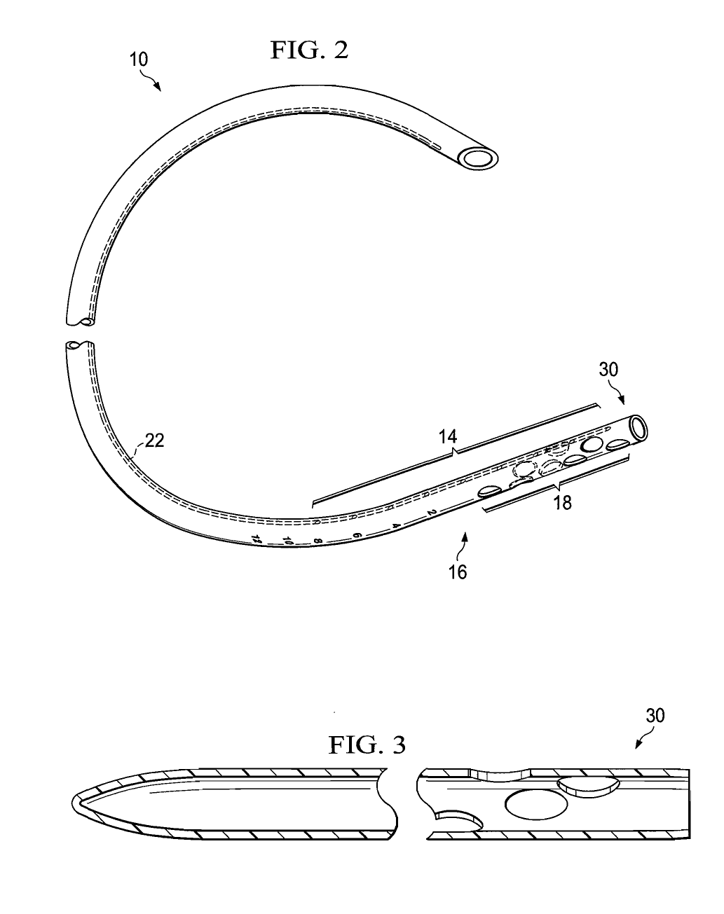

FIG. 2 is a simplified somatic diagram of catheter 10 and, further, illustrates the supple nature of catheter 10. In other example embodiments, catheter 10 is more rigid and cannot be bent in the manner shown in FIG. 2. Note that the radiopaque line offers guidance to a surgeon, where marks (in this example) are provided at every 2 centimeters from the last eye (e.g., for ascertaining the depth of placement). In addition, catheter 10 can have crossed eyes to assist the surgeon. Additionally, catheter 10 can have a proximal end that is fitted with a pull through tongue for easy insertion from within the chest during open operative procedures. In addition, catheter 10 can have a matching size loose connector, which facilitates an easier connection to a drainage system.

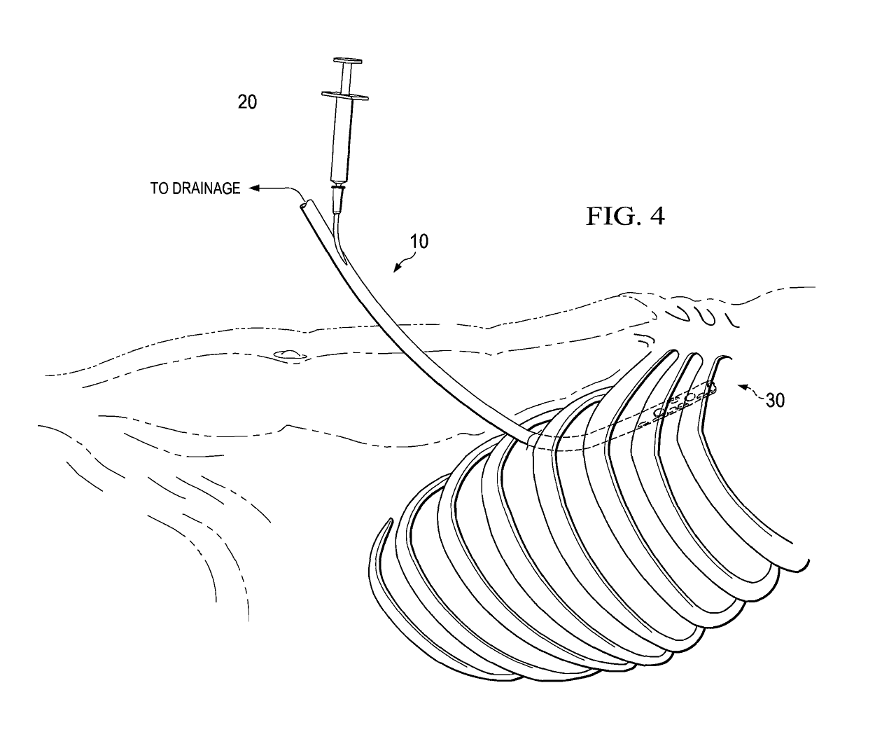

FIG. 3 is a simplified schematic diagram of a tip 30, which in one example, is a smooth and tapered tip that can be easily grasped and maneuvered by a tending physician. Other shapes and dimensions could certainly be used and are clearly within the broad scope of the present invention and its teachings. FIG. 4 is a simplified schematic diagram of catheter 10 in an example patient procedure. Note that just below syringe 20, there is a drainage mechanism for any fluid, gas, or solid (pus, mucus, tissue, etc.) that should leave the body cavity. This drainage mechanism can include pumps, machines, and devices that address the removal of undesirable substances (air, gas, fluid, etc.), in the body.

Note that the preceding FIGURES are only offering a sampling of the myriad of possible applications that involve catheter 10. Other applications could involve other types of procedures: particularly those procedures that involve some type of suction or aspiration (or a pressurized environment). It is also important to note that the stages and steps described above illustrate only some of the possible operations that may be executed by, or within, the present system. Some of these stages and/or steps may be deleted or removed where appropriate, or these stages and/or steps may be modified, enhanced, or changed considerably without departing from the scope of the present invention. In addition, a number of these operations have been described as being executed concurrently with, or in parallel to, one or more additional operations. However, the timing of these operations may be altered.

The preceding example flows have been offered for purposes of teaching and discussion. Substantial flexibility is provided by the tendered system in that any suitable arrangements, chronologies, configurations, and timing mechanisms may be provided without departing from the broad scope of the present invention. Accordingly, any appropriate ancillary structure, component, or device may be included along with catheter 10 to effectuate the tasks and operations of the elements and activities associated with providing an optimal pain management system.

Although the present invention has been described in detail with reference to particular embodiments, it should be understood that various other changes, substitutions, and alterations may be made hereto without departing from the spirit and scope of the present invention. The illustrated device and operations have only been offered for purposes of example and teaching. Suitable alternatives and substitutions are envisioned and contemplated by the present invention: such alternatives and substitutions being clearly within the broad scope of the proposed solutions. In addition, while the foregoing discussion has focused on thoracic and vascular activities, any other suitable environment requiring the need for pain management in the placement of catheters may benefit from the teachings provided herein. It should also be noted that catheter 10 may be constructed of any suitable combination of rubber, silicone, plastic, metal alloy, or any other viable composition that could withstand and readily accommodate the forces as explained herein.

Although the present invention has been described with several embodiments, a myriad of changes, variations, alterations, transformations, and modifications may be suggested to one skilled in the art, and it is intended that the present invention encompass such changes, variations, alterations, transformations, and modifications as fall within the scope of the appended claims.

1. An apparatus, comprising:
a catheter coupled to a channel, wherein the catheter includes a first set of holes in which materials may pass, and wherein the channel defines a pathway that extends along a portion of the catheter, the channel interfacing with a second set of holes that are different from the first set of holes and access to the channel occurs via the second set of holes and not the first set of holes, wherein the second set of holes is configured radially around the catheter such that they are coplanar with each other and perpendicular to the channel, and wherein the channel receives a substance that is delivered to an area close to or at the area in which the channel resides and the substance is delivered via the second set of holes, and wherein the distal end of the catheter is cone-shaped and tapered.

2. The apparatus of claim 1, wherein the second set of holes are shaped as hemispheres, as circles, or as ovals.

3. The apparatus of claim 1, wherein the catheter includes a first marker line having incremental gradations for fluid measurement in the catheter, and a second marker line, which is horizontal and which is configured opposite the first marker line for orientation of the apparatus.

4. The apparatus of claim 1, wherein the second set of holes are spaced equidistant from one another.

5. The apparatus of claim 1, wherein the channel interfaces with a syringe or a pump that delivers the substance.

6. The apparatus of claim 5, wherein the syringe operates as a one-way valve such that the substance can be pushed out of the second set of holes, but egressing materials are prohibited from returning down the channel via the second set of holes.

7. The apparatus of claim 1, wherein the substance is a gas, a fluid, or a solid.

8. The apparatus of claim 1, wherein the catheter is constructed of a polyvinyl chloride (PVC) material.

9. The apparatus of claim 1, wherein the catheter includes a radiopaque marking.

10. The apparatus of claim 1, wherein the second set of holes are spaced equidistant from one another.

11. The apparatus of claim 1, wherein the second set of holes are spaced one centimeter apart from one another.

12. The apparatus of claim 1, wherein the substance is a drug or a pharmaceutical.

13. The apparatus of claim 1, wherein the substance is a local anesthesia.

14. The apparatus of claim 1, wherein the catheter includes a proximal end that is coupled to a mechanism associated with aspiration or suction activities.

15. The apparatus of claim 1, wherein the first set of holes are shaped as 270° outlets.

16. An apparatus, comprising:
a catheter coupled to a channel, wherein the catheter includes a first set of holes in which materials may pass, and wherein the channel defines a pathway that extends along a portion of the catheter, the channel interfacing with a second set of holes that are different from the first set of holes and access to the channel occurs via the second set of holes and not the first set of holes, and wherein the channel receives a substance that is delivered to an area close to or at the area in which the channel resides and the substance is delivered via the second set of holes, and wherein a distal end of the catheter is rounded and tapered, the channel interfacing with a syringe that delivers the substance, wherein the second set of holes is configured radially around the catheter such that they are coplanar with each other and perpendicular to the channel, and wherein the distal end of the catheter is cone-shaped and tapered.

17. The apparatus of claim 16, wherein the second set of holes are shaped as hemispheres, and wherein the second set of holes are spaced equidistant from one another.

18. The apparatus of claim 16, wherein the syringe operates as a one-way valve such that the substance can be pushed out of the second set of holes, but egressing materials are prohibited from returning to the channel.

19. The apparatus of claim 16, wherein the catheter is constructed of a polyvinyl chloride (PVC) material and the catheter includes a radiopaque marking.

20. The apparatus of claim 16, wherein the second set of holes are spaced equidistant from one another and the second set of holes are spaced one centimeter apart from one another, and wherein the first set of holes are shaped as 270° outlets.
